SEQUENCE LISTING (37 sequences)

SEQ ID NO: 1 - protein, 289 aa, Homo sapiens
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 2 - protein, 176 aa, Homo sapiens
MEAWECLEDFSAVRNLLEQSSNSTSWFWRFLWGSSQAKLVCRIKEDYKWEFEELLKSCGELFDSLNLGHQALFQEKVIKTLDFSTPGRAAAAVAFLSFIKDKWSEETHLSGGYLLDFLAMHLWRAVVRHKNRLLLLSSVRPAIIPTEEQQQQQEEARRRRQEQSPWNPRAGLDPRE

SEQ ID NO: 3 - protein, 496 aa, Homo sapiens
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 4 - protein, 140 aa, Homo sapiens
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAV

SEQ ID NO: 5 - protein, 449 aa, Homo sapiens
METRGRRPAALQHQQDQPQAHPGQRAARSAPLHRDPDYADEDPAPVERHDPGPSGRAPTTAVQRKPPQPAKRGDMLDRDAVEQVTELWDRLELLGQTLKSMPTADGLKPLKNFASLQELLSLGGERLLADLVRENMRVRDMLNEVAPLLRDDGSCSSLNYQLHPVIGVIYGPTGCGKSQLLRNLLSSQLISPTPETVFFIAPQVDMIPPSELKAWEMQICEGNYAPGPDGTIIPQSGTLRPRFVKMAYDDLILEHNYDVSDPRNIFAQAAARGPIAIIMDECMENLGGHKGVSKFFHAFPSKLHDKFPKCTGYTVLVVLHNMNPRRDMAGNIANLKIQSKMHLISPRMHPSQLNRFVNTYTKGLPLAISLLLKDIFRHHAQRSCYDWIIYNTTPQHEALQWCYLHPRDGLMPMYLNIQSHLYHVLEKIHRTLNDRDRWSRAYRARKTPK

SEQ ID NO: 6 - protein, 1198 aa, Homo sapiens
MALAQAHRARRLHAEAPDSGDQPPRRRVRQQPTRAAPAPARARRRRAPAPSPGGSGAPPTSGGSPASPLLDASSKDTPAAHRPPRGTVVAPRGCGLLQAIDAATNQPLEIRYHLDLARALTRLCEVNLQELPPDLTPRELQTMDSSHLRDVVIKLRPPRADIWTLGSRGVVVRSTVTPLEQPDGQGQAAEVEDHQPNPPGEGLKFPLCFLVRGRQVNLVQDVQPVHRCQYCARFYKSQHECSARRRDFYFHHINSHSSNWWREIQFFPIGSHPRTERLFVTYDVETYTWMGAFGKQLVPFMLVMKFGGDEPLVTAARDLAANLGWDRWEQDPLTFYCITPEKMAIGRQFRTFRDHLQMLMARDLWSSFVASNPHLADWALSEHGLSSPEELTYEELKKLPSIKGIPRFLELYIVGHNINGFDEIVLAAQVINNRSEVPGPFRITRNFMPRAGKILFNDVTFALPNPRSKKRTDFLLWEQGGCDDTDFKYQYLKVMVRDTFALTHTSLRKAAQAYALPVEKGCCAYQAVNQFYMLGSYRSEADGFPIQEYWKDREEFVLNRELWKKKGQDKYDIIKETLDYCALDVQVTAELVNKLRDSYASFVRDAVGLTDASFNVFQRPTISSNSHAIFRQIVFRAEQPARSNLGPDLLAPSHELYDYVRASIRGGRCYPTYLGILREPLYVYDICGMYASALTHPMPWGPPLNPYERALAARAWQQALDLQGCKIDYFDARLLPGVFTVDADPPDETQLDPLPPFCSRKGGRLCWTNERLRGEVATSVDLVTLHNRGWRVHLVPDERTTVFPEWRCVAREYVQLNIAAKERADRDKNQTLRSIAKLLSNALYGSFATKLDNKKIVFSDQMDAATLKGITAGQVNIKSSSFLETDNLSAEVMPAFQREYSPQQLALADSDAEESEDERAPTPFYSPPSGTPGHVAYTYKPITFLDAEEGDMCLHTLERVDPLVDNDRYPSHLASFVLAWTRAFVSEWSEFLYEEDRGTPLEDRPLKSVYGDTDSLFVTERGHRLMETRGKKRIKKHGGNLVFDPERPELTWLVECETVCGACGADAYSPESVFLAPKLYALKSLHCPSCGASSKGKLRAKGHAAEGLDYDTMVKCYLADAQGEDRQRFSTSRTSLKRTLASAQPGAHPFTVTQTTLTRTLRPWKDMTLARLDEHRLLPYSESRPNPRNEEICWIEMP

SEQ ID NO: 7 - protein, 671 aa, Homo sapiens
MALSVNDCARLTGQSVPTMEHFLPLRNIWNRVRDFPRASTTAAGITWMSRYIYGYHRLMLEDLAPGAPATLRWPLYRQPPPHFLVGYQYLVRTCNDYVFDSRAYSRLRYTELSQPGHQTVNWSVMANCTYTINTGAYHRFVDMDDFQSTLTQVQQAILAERVVADLALLQPMRGFGVTRMGGRGRHLRPNSAAAAAIDARDAGQEEGEEEVPVERLMQDYYKDLRRCQNEAWGMADRLRIQQAGPKDMVLLSTIRRLKTAYFNYIISSTSARNNPDRRPLPPATVLSLPCDCDWLDAFLERFSDPVDADSLRSLGGGVPTQQLLRCIVSAVSLPHGSPPPTHNRDMTGGVFQLRPRENGRAVTETMRRRRGEMIERFVDRLPVRRRRRRVPPPPPPPEEEEGEALMEEEIEEEEEAPVAFEREVRDTVAELIRLLEEELTVSARNSQFFNFAVDFYEAMERLEALGDINESTLRRWVMYFFVAEHTATTLNYLFQRLRNYAVFARHVELNLAQVVMRARDAEGGVVYSRVWNEGGLNAFSQLMARISNDLAATVERAGRGDLQEEEIEQFMAEIAYQDNSGDVQEILRQAAVNDTEIDSVELSFRLKLTGPVVFTQRRQIQEINRRVVAFASNLRAQHQLLPARGADVPLPPLPAGPEPPLPPGARPRHRF

SEQ ID NO: 8 - protein, 415 aa, Homo sapiens
MHPVLRQMRPPPQQRQEQEQRQTCRAPSPPPTASGGATSAVDAAADGDYEPPRRRARHYLDLEEGEGLARLGAPSPERYPRVQLKRDTREAYVPRQNLFRDREGEEPEEMRDRKFHAGRELRHGLNRERLLREEDFEPDARTGISPARAHVAAADLVTAYEQTVNQEINFQKSFNNHVRTLVAREEVAIGLMHLWDFVSALEQNPNSKPLMAQLFLIVQHSRDNEAFRDALLNIVEPEGRWLLDLINILQSIVVQERSLSLADKVAAINYSMLSLGKFYARKIYHTPYVPIDKEVKIEGFYMRMALKVLTLSDDLGVYRNERIHKAVSVSRRRELSDRELMHSLQRALAGTGSGDREAESYFDAGADLRWAPSRRALEAAGAGPGLAVAPARAGNVGGVEEYDEDDEYEPEDGEY

SEQ ID NO: 9 - protein, 585 aa, Homo sapiens
MMQDATDPAVRAALQSQPSGLNSTDDWRQVMDRIMSLTARNPDAFRQQPQANRLSAILEAVVPARANPTHEKVLAIVNALAENRAIRPDEAGLVYDALLQRVARYNSGNVQTNLDRLVGDVREAVAQRERAQQQGNLGSMVALNAFLSTQPANVPRGQEDYTNFVSALRLMVTETPQSEVYQSGPDYFFQTSRQGLQTVNLSQAFKNLQGLWGVRAPTGDRATVSSLLTPNSRLLLLLIAPFTDSGSVSRDTYLGHLLTLYREAIGQAHVDEHTFQEITSVSRALGQEDTGSLEATLNYLLTNRRQKIPSLHSLNSEEERILRYVQQSVSLNLMRDGVTPSVALDMTARNMEPGMYASNRPFINRLMDYLHRAAAVNPEYFTNAILNPHWLPPPGFYTGGFEVPEGNDGFLWDDIDDSVFSPQPQTLLELQQREQAEAALRKESFRRPSSLSDLGAAAPRSDASSPFPSLIGSLTSTRTTRPRLLGEEEYLNNSLLQPQREKNLPPAFPNNGIESLVDKMSRWKTYAQEHRDVPGPRPPTRRQRHDRQRGLVWEDDDSADDSSVLDLGGSGNPFAHLRPRLGRMF

SEQ ID NO: 10 - protein, 571 aa, Homo sapiens
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 11 - protein, 198 aa, Homo sapiens
MSILISPSNNTGWGLRFPSKMFGGAKKRSDQHPVRVRGHYRAPWGAHKRGRTGRTTVDDAIDAVVEEARNYTPTPPPVSTVDAAIQTVVRGARRYAKMKRRRRRVARRHRRRPGTAAQRAAAALLNRARRTGRRAAMRAARRLAAGIVTVPPRSRRRAAAAAAAAISAMTQGRRGNVYWVRDSVSGLRVPVRTRPPRN

SEQ ID NO: 12 - protein, 368 aa, Homo sapiens
MSKRKIKEEMLQVIAPEIYGPPKKEEQDYKPRKLKRVKKKKKDDDDELDDEVELLHATAPRRRVQWKGRRVKRVLRPGTTVVFTPGERSTRTYKRVYDEVYGDEDLLEQANERLGEFAYGKRHKDMLALPLDEGNPTPSLKPVTLQQVLPALAPSEEKRGLKRESGDLAPTVQLMVPKRQRLEDVLEKMTVEPGLEPEVRVRPIKQVAPGLGVQTVDVQIPTTSSTSIATATEGMETQTSPVASAVADAAVQAVAAAASKTSTEVQTDPWMFRVSAPRRPRGSRKYGAASALLPEYALHPSIAPTPGYRGYTYRPRRRATTRRRTTTGTRRRRRRRQPVLAPISVRRVAREGGRTLVLPTARYHPSIV

SEQ ID NO: 13 - protein, 80 aa, Homo sapiens
MALTCRLRFPVPGFRGRMHRRRGMAGHGLTGGMRRAHHRRRRASHRRMRGGILPLLIPLIAAAIGAVPGIASVALQAQRH

SEQ ID NO: 14 - protein, 250 aa, Homo sapiens
MEDINFASLAPRHGSRPFMGNWQDIGTSNMSGGAFSWGSLWSGIKNFGSTVKNYGSKAWNSSTGQMLRDKLKEQNFQQKVVDGLASGISGVVDLANQAVQNKINSKLDPRPPVEEPPPAVETVSPEGRGEKRPRPDREETLVTQIDEPPSYEEALKQGLPTTRPIAPMATGVLGQHTPVTLDLPPPADTQQKPVLPGPTAVVVTRPSRASLRRAASGPRSLRPVASGNWQSTLNSIVGLGVQSLKRRRCF

SEQ ID NO: 15 - protein, 952 aa, Homo sapiens
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 16 - protein, 204 aa, Homo sapiens
MGSSEQELKAIVKDLGCGPYFLGTYDKRFPGFVSPHKLACAIVNTAGRETGGVHWMAFAWNPHSKTCYLFEPFGFSDQRLKQVYQFEYESLLRRSAIASSPDRCITLEKSTQSVQGPNSAACGLFCCMFLHAFANWPQTPMDHNPTMNLITGVPNSMLNSPQVQPTLRRNQEQLYSFLERHSPYFRSHSAQIRSATSFCHLKNM

SEQ ID NO: 17 - protein, 529 aa, Homo sapiens
MASREEEQRETTPERGRGAARRPPTMEDVSSPSPSPPPPRAPPKKRMRRRIESEDEEDSSQDALVPRTPSPRPSTSAADLAIAPKKKKKRPSPKPERPPSPEVIVDSEEEREDVALQMVGFSNPPVLIKHGKGGKRTVRRLNEDDPVARGMRTQEEEEEPSEAESEITVMNPLSVPIVSAWEKGMEAARALMDKYHVDNDLKANFKLLPDQVEALAAVCKTWLNEEHRGLQLTFTSNKTFVTMMGRFLQAYLQSFAEVTYKHHEPTGCALWLHRCAEIEGELKCLHGSIMINKEHVIEMDVTSENGQRALKEQSSKAKIVKNRWGRNVVQISNTDARCCVHDAACPANQFSGKSCGMFFSEGAKAQVAFKQIKAFMQALYPNAQTGHGHLLMPLRCECNSKPGHAPFLGRQLPKLTPFALSNAEDLDADLISDKSVLASVHHPALIVFQCCNPVYRNSRAQGGGPNCDFKISAPDLLNALVMVRSLWSENFTELPRMVVPEFKWSTKHQYRNVSLPVAHSDARQNPFDF

SEQ ID NO: 18 - protein, 807 aa, Homo sapiens
MESVEKKDSLTAPSEFATTASTDAANAPTTFPVEAPPLEEEEVIIEQDPGFVSEDDEDRSVPTEDKKQDQDNAEANEEQVGRGDERHGDYLDVGDDVLLKHLQRQCAIICDALQERSDVPLAIADVSLAYERHLFSPRVPPKRQENGTCEPNPRLNFYPVFAVPEVLATYHIFFQNCKIPLSCRANRSRADKQLALRQGAVIPDIASLNEVPKIFEGLGRDEKRAANALQQENSENESHSGVLVELEGDNARLAVLKRSIEVTHFAYPALNLPPKVMSTVMSELIVRRAQPLERDANLQEQTEEGLPAVGDEQLARWLQTREPADLEERRKLMMAAVLVTVELECMQRFFADPEMQRKLEETLHYTFRQGYVRQACKISNVELCNLVSYLGILHENRLGQNVLHSTLKGEARRDYVRDCVYLFLCYTWQTAMGVWQQCLEECNLKELQKLLKQNLKDLWTAFNERSVAAHLADIIFPERLLKTLQQGLPDFTSQSMLQNFRNFILERSGILPATCCALPSDFVPIKYRECPPPLWGHCYLLQLANYLAYHSDIMEDVSGDGLLECHCRCNLCTPHRSLVCNSQLLNESQIIGTFELQGPSPDEKSAAPGLKLTPGLWTSAYLRKFVPEDYHAHEIRFYEDQSRPPNAELTACVITQGHILGQLQAINKARQEFLLRKGRGVYLDPQSGEELNPIPPPPQPYQQQPRALASQDGTQKEAAAAAATHGRGGILGQSGRGGFGRGGGGHDGRLGEPRRGSFRGRRGVRRNTVTLGRIPLAGAPEIGNRFQHGYNLRSSGAAGTARSPTQP

SEQ ID NO: 19 - protein, 229 aa, Homo sapiens
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAAHTAPAAAAAAATAAATQKQRRPDSKTLTKPKKSTAAAAAGGGALRLAPNEPVSTRELRNRIFPTLYAIFQQSRGQEQELKIKNRSLRSLTRSCLYHKSEDQLRRTLEDAEALFSKYCALTLKD

SEQ ID NO: 20 - protein, 196 aa, Homo sapiens
MAPKKKLQLPPPPTDEEEYWDSQAEEVLDEEEEDMMEDWESLDEEASEVEEVSDETPSPSVAFPSPAPQKSATGSSMATTSAPQAPPALPVRRPNRRWDTTGTRAGKSKQPPPLAQEQQQRQGYRSWRGHKNAIVACLQDCGGNISFARRFLLYHHGVAFPRNILHYYRHLYSPYCTGGSGSGSNSSGHTEAKATG

SEQ ID NO: 21 - protein, 227 aa, Homo sapiens
MSKEIPTPYMWSYQPQMGLAAGAAQDYSTRINYMSAGPHMISRVNGIRAHRNRILLEQAAITTTPRNNLNPRSWPAALVYQESPAPTTVVLPRDAQAEVQMTNSGAQLAGGFRHRVRSPGQGITHLTIRGRGIQLNDESVSSSLGLRPDGTFQIGGAGRPSFTPRQAILTLQTSSSEPRSGGIGTLQFIEEFVPSVYFNPFSGPPGHYPDQFIPNFDAVKDSADGYD

SEQ ID NO: 22 - protein, 107 aa, Homo sapiens
MLSGEAEQLRLKHLVHCRRHKCFARDSGEFCYFELPEDHIEGPAHGVRLTAQGELARSLIREFTQRPLLVERDRGPCVLTVICNCPNLGLHQDLCCHLCAEYNKYRN

SEQ ID NO: 23 - protein, 63 aa, Homo sapiens
MNNSSNSTGYSNSGFSRIGVGVILCLVILFILILTLLCLRLAACCVHICIYCQLFKRWGRHPR

SEQ ID NO: 24 - protein, 160 aa, Homo sapiens
MIRYIILGLLTLASAHGTTQKVDFKEPACNVTFAAEANECTTLIKCTTEHEKLLIRHKNKIGKYAVYAIWQPGDTTEYNVTVFQGKSHKTFMYTFPFYEMCDITMYMSKQYKLWPPQNCVENTGTFCCTAMLITVLALVCTLLYIKYKSRRSFIEEKKMP

SEQ ID NO: 25 - protein, 93 aa, Homo sapiens
MTNTTNAAAATGLTSTTNTPQVSAFVNNWDNLGMWWFSIALMFVCLIIMWLICCLKRKRARPPIYSPIIVLHPNNDGIHRLDGLKHMFFSLTV

SEQ ID NO: 26 - protein, 91 aa, Homo sapiens
MIPRVFILLTLVALFCACSTLAAVSHIEVDCIPAFTVYLLYGFVTLTLICSLITVVIAFIQCIDWVCVRFAYLRHHPQYRDRTIAELLRIL

SEQ ID NO: 27 - protein, 132 aa, Homo sapiens
MKFTVTFLLIICTLSAFCSPTSKPQRHISCRFTRIWNIPSCYNEKSDLSEAWLYAIISVMVFCSTILALAIYPYLDIGWKRIDAMNHPTFPAPAMLPLQQVVAGGFVPANQPRPTSPTPTEISYFNLTGGDD

SEQ ID NO: 28 - protein, 128 aa, Homo sapiens
MTDTLDLEMDGIITEQRLLERRRAAAEQQRMNQELQDMVNLHQCKRGIFCLVKQAKVTYDSNTTGHRLSYKLPTKRQKLVVMVGEKPITITQHSVETEGCIHSPCQGPEDLCTLIKTLCGLKDLIPFN

SEQ ID NO: 29 - protein, 54 aa, Homo sapiens
MKIVGADGQEQEETDIPFRLWRKFAARRKLQYQSWEEGKEVLLNKLDRNLLTDF

SEQ ID NO: 30 - protein, 581 aa, Homo sapiens
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 31 - protein, 150 aa, Homo sapiens
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 32 - protein, 294 aa, Homo sapiens
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 33 - protein, 114 aa, Homo sapiens
MVLPALPAPPVCDSQNECVGWLGVAYSAVVDVIRAAAHEGVYIEPEARGRLDALREWIYYNYYTERSKRRDRRRRSVCHARTWFCFRKYDYVRRSIWHDTTTNTISVVSAHSVQ

SEQ ID NO: 34 - protein, 116 aa, Homo sapiens
MIRCLRLKVEGALEQIFTMAGLNIRDLLRDILRRWRDENYLGMVEGAGMFIEEIHPEGFSLYVHLDVRAVCLLEAIVQHLTNAIICSLAVEFDHATGGERVHLIDLHFEVLDNLLE

SEQ ID NO: 35 - protein, 130 aa, Homo sapiens
MFERKMVSFSVVVPELTCLYLHEHDYDVLSFLREALPDFLSSTLHFISPPMQQAYIGATLVSIAPSMRVIISVGSFVMVPGGEVAALVRADLHDYVQLALRRDLRDRGIFVNVPLLNLIQVCEEPEFLQS

SEQ ID NO: 36 - protein, 128 aa, Homo sapiens
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 37 - DNA, 35938 bp, Homo sapiens
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

(19) United States
(12) Patent Application Publication
(10) Pub. No.: US 2009/0253184 A1
(43) Pub. Date: Oct. 8, 2009

(54) COMPOSITIONS AND METHODS RELATED TO AN ADENOVIRAL TRANS-COMPLEMENTING CELL LINE

(75) Inventors: Peter Clarke, Lincroft, NJ (US); Shuyuan Zhang, Boyds, MD (US); Hai Pham, Houston, TX (US); Joe Senesac, Norristown, PA (US)

Correspondence Address: FULBRIGHT & JAWORSKI L.L.P., 600 CONGRESS AVE., SUITE 2400, AUSTIN, TX, 78701, US

(73) Assignee: Introgen Therapeutics, Inc., Houston, TX (US)

(21) Appl. No.: 12/358,662

(22) Filed: Jan. 23, 2009

Related U.S. Application Data
(60) Provisional application No. 61/022,875, filed on Jan. 23, 2008.

(51) Int. Cl. C12N 15/64; C07H 21/00; C12N 5/00

(52) U.S. Cl. 435/91.4; 536/23.72; 435/325

(57) ABSTRACT

Embodiments of the invention include E1 expressing cell lines that can be used in a variety of methods for production of an E1 defective adenovirus. In certain aspects a cell of the invention can be adapted to various culture conditions, e.g., suspension culture in serum free medium. In a further aspect, the cell lines allow isolation and subculture of E1-deleted recombinant adenoviruses in an environment free of replication competent adenovirus (RCA).

This application claims priority to U.S. Provisional Patent application Ser. No. 61/022,875 filed Jan. 23, 2008, entitled “Compositions and Methods Related to an Adenoviral Trans-Complementing Cell Line,” which is incorporated herein by reference in its entirety.

### BACKGROUND OF THE INVENTION

I. Field of the Invention

Embodiments of this invention are directed generally to virology and therapy. In certain aspects the invention is related to a cell lines and related methods for production of adenovirus.

II. Background

Recombinant adenoviruses have been described as useful for delivery of transgenes to cells for a variety of purposes, including both therapeutic and prophylactic (vaccine) uses. However, successful commercialization of E1-deleted adenoviruses will require suitable manufacturing processes. Infection of an E1 trans-complementing cell line with the vector and purification of the resulting lysate is a simple and scalable process that yields sufficient quantities of product. Production of E1-deleted adenovirus vectors for gene therapy has been associated with production of replication competent adenovirus (RCA) caused by homologous recombination between the vector and transfected E1 gene.

Several strategies have been described to avoid RCA. Imler et al., Gene Ther., 3:75-84 (1996) describes an A549 cell stably transfected with E1a and E1b open reading frames (ORFs) and contiguous pIX gene. The E1a was driven by phosphoglycerate kinase promoter and RCA was reportedly eliminated. However, more recent publications describing this system reveal that Imler was unable to detect E1b protein expression. See WO 97/00326.

Another system for avoiding RCA is described in U.S. Pat. No. 5,891,690. The patent describes an Ad E1-complementing cell line having a stably integrated complementation element comprising a portion of the Ad E1 region covering the E1a gene and the E1b gene, but lacking the 5′ ITR, the packaging sequence, and the E1a promoter. Further, the E1a gene is under control of a first promoter element and the E2b gene is under control of a second promoter element. A specific cell line described and claimed nucleotides 532-3525 of Ad5, which includes E1a, the E1b promoter, and a portion of the E1b gene. This cell line does not contain the carboxy terminus of the E1b gene, which encodes the 8.3 kb product, nor does it contain pIX gene sequences.

Additional compositions and methods for producing high yields of E1-defective adenoviruses in the absence of detectable RCA are needed.

### SUMMARY OF THE INVENTION

The present invention provides E1 expressing cell lines that can be used in a variety of methods for production of adenovirus. In certain aspects a cell of the invention can be adapted to various culture conditions, e.g., suspension culture in serum free medium. In a further aspect, the cell lines allow isolation and subculture of E1-deleted recombinant adenoviruses in an environment free of replication competent adenovirus (RCA).

Embodiments of the invention include E1-complementing cell lines stably transformed with one or more nucleic acid molecules or nucleic acid segments encoding adenovirus E1a and adenovirus E1b under the control of a promoter.

In another aspect, the invention provides a method for packaging of E1-defective adenoviral particles in the absence of significant or undetectable amounts of replication competent adenovirus. The method involves introducing an adenoviral vector into E1-complementing cell lines of the invention, where the vector contains a defect in one or more of adenovirus E1 region, adenovirus 5′ and 3′ cis-elements necessary for replication and packaging, adenovirus pIX, and/or regulatory sequences necessary for expression of the adenoviral genes and transgene.

In another aspect, the invention provides a method of producing E1-defective adenoviral particles using the cells of the invention. In a further aspect, the adenoviral particles are substantially free (that is no detectable RCA) or significantly free (acceptable levels of RCA are detected), or absolutely free of replication competent adenovirus. The method involves infecting E1-complementing cell lines of the invention with an E1-defective adenovirus and culturing under conditions which permit the cell to express the E1a and E1b proteins.

Further embodiments of the invention include an expression cassette designed to complement E1 deleted adenoviral vectors. The expression cassette can be transfected into a selected cell line and maintained as an episomal expression cassette or plasmid, or integrated into the genome of a cell. In certain aspects, both the cell line and the adenoviral vector will include protein IX for improved virus yield and stability. The expression cassette designed to be transfected into the selected cell line will be constructed in a manner that will reduce the potential for recombination between the adenoviral vector and the cellular genome or E1 expression cassette. Current expression cassettes within producer cells designed to trans-complement adenoviral vector genes contain an inverted terminal repeat (ITR), the adenoviral E1 gene and the protein IX gene in succession. Likewise, the adenoviral vector genome contains an ITR, a transgene in place of the E1 region, and protein IX gene in succession. However, in the contemplated cell line, the expression cassette will have a non-native promoter in place of the ITR, followed by a spacer sequence and the protein IX gene. The spacer sequence can be 1, 2, 3, 4, 5, 6, 7, 8, 9, 10, 50, 100, 200, 400, 800 kilobases (kb) or more in length, including all ranges and values there between. In a further aspect, due to the degeneracy of the genetic code, the segments of the expression cassette, including but not limited to the protein IX gene encoding segments, will be constructed such that the nucleic acid sequence is not homologous or not identical to the corresponding adenoviral vector gene encoding segments yet still allow for function and/or the production of an encoded protein. Recombination event that could generate a replication competent adenovirus will be reduced, lessened or eliminated by one or more of (1) a lack of homology between the non native promoter of the expression cassette and the ITR of the adenoviral vector, (2) the spacing gap between the expression cassette E1 gene and the protein IX gene as compared to the adenoviral vector, and/or (3) the lack of nucleic acid homology between the cassette protein IX gene and the vector protein IX gene. The cells used for generating this producer cell line can be, but are not limited to primary human tonsil or umbilical cord cells, HeLa cells or other stable or primary cell lines.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B. Shows a schematic of one example of an expression cassette to be used in generating an E1 complementing cell line.

### DETAILED DESCRIPTION OF THE INVENTION

Like other biological manufacturing systems, a key hurdle in the development of adenovirus-based products is the integration of product safety attributes with commercial scale production. The inventors believe these issues can be addressed through the use of an appropriate host cell line for the propagation of the adenovirus in the manufacturing process. By creating a cell line capable of generating a safe and robust product, the methods and composition described herein will increase the ability of adenoviral products to meet the emerging potential of the adenovirus delivery system in gene therapy and vaccines. To that end, a cell line will overcome one or more of the following hurdles in order to generate a safe and economical adenovirus-based product: (1) traceability, (2) free of animal-derived components, (3) scalable, and (4) free of replication-competent adenovirus (RCA).

Traceability is a safety feature advocated by the FDA but is seldom achieved. Traceability refers to the ability to precisely trace the lineage of a given cell line and to enumerate the materials that it has been exposed to. For instance, many of the cell lines used today to manufacture gene therapies, protein, and antibodies have originated from the American Type Culture Collection (ATCC). ATCC's widespread use has unfortunately contributed to the often poor or untraceable origins of its more popular cell banks, as deposits to ATCC do not provide complete traceability and material usage histories. It would be one advantage if a cell line has a well defined and demonstrated profile of the cell lines' origins and materials utilized in its establishment to ensure the integrity of the product and its commercial use.

The characteristic of being free of animal-derived components is another area the pharmaceutical industry has focused on to improve product safety. Typically, this process has been centered on the development of human-originating materials. For instance, humanization of antibodies has evolved as the status quo due to concerns over the potential differential antigenicity of animal-derived antibodies. While the jury may still be out on the validity of these claims, it is believe a similar trend has emerged in the adenoviral product market. Further, it is believed that the trend toward human-based reagents may extend to the FDA's opinion on potential products for commercialization. In light of these trends, a human-derived cell line would be of benefit.

The competitive manufacturing of any drug product relies on the ability of the manufacturer to produce sufficient product at a reasonable cost. In adenovirus manufacturing the ability to generate commercial scale lots at a competitive cost will rely on cell lines capable of being grown in suspension and at increased volumes. Introgen currently relies on cancer-derived (HeLa) and transformed (293) cell lines because of their reasonably rapid growth characteristics and their adaptability to animal serum-free, suspension environments. However, it is believe that the use of adenovirus in vaccines, for instance, will require a more rigorous safety profile while maintaining the growth potential of current cell lines. Another consideration in the generation of a cell line is the use of normal or cancerous cells. Typically, cancer cell lines are used because of the cells' ability to grow at a tremendous rate and indefinitely. However, the use of a diseased cell may compromise the safety of the resultant product and instead believe that the use of a cell line originating from a normal cell will continue to be a preferred source as the industry matures.

Finally, replication competent adenovirus (or RCA) generation is a common problem in the manufacture of adenovirus products that utilize E1 complementation. RCA occurs when recombination events due to sequence homology between the adenoviral vector and cell genomic DNA result in an adenoviral DNA sequence that can replicate in the absence of E1 complementation. The current standard E1 complementing cell line, 293 HEK, causes RCA generation at very small but measurable levels when appropriate viral construction techniques are used. Since RCA may increase the immunogenicity of the adenovirus product and is highly desirable to eliminate its presence in the adenovirus products particularly for vaccine applications. By careful design of the E1 expression cassette in the selected cell line to eliminate sequence homology with available adenovirus vectors, its expect generation of RCA can be eliminated during adenovirus production using the new cell line.

The adenovirus is an attractive delivery system because of its high gene transfer efficiency and can be produced to a high titer and purity. Currently, suspension cell processes with average yields of 1×10¹⁶ viral particles per batch are used. The cell growth media and other process components are typically free of protein, serum, and animal-derived components making it suitable for a broad range of both prophylactic and therapeutic vaccine products. It is further contemplated that the cell line will have future use in a variety of commercial biologics including vaccines, therapeutic proteins, and antibodies.

Other useful cell lines may be derived from a cell line of the invention. For example, a cell line may be modified to stably express another desired protein(s) using the techniques described herein, as well as techniques known in the art. In one embodiment, a derivative cell line may contain one or more sequences expressing adenoviral proteins (or functional fragments thereof) including, but not limited to E2 (E2a and/or E2b), E3 and/or any coding region of E4, e.g., ORF6. Thus, in one embodiment, a derivative cell line may be produced which expresses the required functions of the E2 region or E4 region, or combinations thereof. Suitably, the nucleic acid molecule(s) used to produce the derivative cell line contains no adenoviral sequences 5′ to the E1 coding region and only the minimal adenoviral sequences required to express the desired functional proteins in the host cell. Given this information, one of skill in the art may readily engineer other derivative cell lines.

### I. E1-COMPLEMENTING CELL LINE

Cell lines or primary cells can be transformed with an expression cassette to produce a cell or cell line of the invention resulting in an E1-trans-complementing cell line(s). A precursor to the E1-trans-complementing cell line can be selected from any mammalian species, such as human cell types, including without limitation, cells such as primary cells isolated from various human tissues, e.g., human tonsil or umbilical cord cells; cell lines such as HeLa, Vero, A549 and/or HKB cells or other human cell lines. Other mammalian species cells are also useful, for example, primate cells, rodent cells or other cells commonly used in biological laboratories. The selection of the mammalian species providing the cells is not a limitation of this invention; nor is the type of mammalian cell, i.e., fibroblast, hepatocyte, tumor cell, etc.

Suitably, the target cells are transformed with a nucleic acid, e.g. an expression cassette, comprising nucleic acid sequences encoding adenovirus E1a and E1b under the control of a heterologous promoter. In certain aspects, the expression cassette will have a non-native promoter in place of the ITR, followed by a spacer sequence and the protein IX gene. The spacer sequence can be 1, 2, 3, 4, 5, 6, 7, 8, 9, 10, 50, 100, 200, 400, 800 kilobases (kb) or more in length, including all ranges and values there between. This molecule lacks adenoviral sequences 5′ of the E1 region, preferably excluding the native E1a promoter and contains minimal sequences 3′ to the E1 region.

The DNA sequences encoding the adenovirus E1a and E1b genes useful in this invention may be selected from among any known adenovirus type, including the presently identified 41 human types. Similarly, adenoviruses known to infect other animals may supply the gene sequences. The selection of the adenovirus type for each E1a and E1b gene sequence does not limit this invention. The sequences for a number of adenovirus serotypes, including that of serotype Ad5, are available from Genbank and incorporated herein by reference as of the filing date of this application. A variety of adenovirus strains are available from the ATCC, or are available by request from a variety of commercial and institutional sources. In the following exemplary embodiment the E1a and E1b gene sequences are those from adenovirus serotype 5 (Ad5).

By “nucleic acid that expresses the E1a gene product,” it is meant any adenovirus gene encoding E1a protein (including proteins that are 85, 90, 91, 92, 93, 94, 95, 96, 97, 98, 99% or more identical in amino acid sequence) or any functional E1a polypeptide segment thereof. Similarly included are any alleles or other modifications of the E1a gene or functional portion. Such modifications may be deliberately introduced by resort to conventional genetic engineering or mutagenic techniques to enhance the E1a expression or function in some manner, as well as naturally occurring allelic variants thereof. The nucleic acid sequence may be modified to reduce the identity.

By “nucleic acid that expresses the E1b gene product,” it is meant any adenovirus gene encoding E1b (including proteins that are 85, 90, 91, 92, 93, 94, 95, 96, 97, 98, 99% or more identical in amino acid sequence) or any functional E1b portion. Similarly included are any alleles or other modifications of the E1b gene or functional portion. Such modifications may be deliberately introduced by resort to conventional genetic engineering or mutagenic techniques to enhance the E1b expression or function in some manner, as well as naturally occurring allelic variants thereof.

The nucleic acid molecule carrying the Ad E1a and Ad E1b may be in any form which transfers these components to the host cell. Most suitably, these sequences are contained within an expression cassette or an expression vector. An “expression cassette” includes a polynucleotide that includes all elements for expression, such as a promoter and a poly-adenylation site. An “expression vector” includes, without limitation, any genetic element, such as a plasmid, phage, transposon, cosmid, chromosome, virus, virion, etc. that include elements for propagation, insertion, or other functions not directly related to expression of a coding region. In one aspect, the nucleic acid molecule is a plasmid carrying Ad E1a, Ad E1b, and pIX sequences under the control of a heterologous promoter, that is a promoter that is not the typical promoter used by adenovirus to express the E1a and/or E1b regions.

The nucleic acid molecule may contain other non-viral sequences, such as those encoding certain selectable reporters or marker genes, e.g., sequences encoding hygromycin or purimycin, or the neomycin resistance gene for G418 selection, among others. The molecule may further contain other components.

Conventional techniques may be utilized for construction of the nucleic acid molecules of the invention. See, generally, Sambrook et al., Molecular Cloning: A Laboratory Manual, Cold Spring Harbor Laboratories, Cold Spring Harbor, N.Y.

Once the desired nucleic acid molecule is engineered, it may be transferred to the target cell by any suitable method. Such methods include, for example, transfection, electroporation, liposome delivery, membrane fusion techniques, high velocity DNA-coated pellets, viral infection and protoplast fusion. Thereafter, cells are cultured according to standard methods and, optionally, seeded in media containing an antibiotic to select for cells containing the cells expressing the resistance gene. After a period of selection, the resistant colonies are isolated, expanded, and screened for E1 expression. See, Sambrook et al., cited above.

Promoters and Enhancers—In order for the expression cassette to effect expression of complementing components, the nucleic acid encoding regions will be under the transcriptional control of a promoter. A “promoter” is a control sequence that is a region of a nucleic acid sequence at which initiation and rate of transcription are controlled. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Any promoter known to those of ordinary skill in the art that would be active in a complementing cell is contemplated as a promoter that can be applied in the methods and compositions of the present invention. One of ordinary skill in the art would be familiar with the numerous types of promoters that can be applied in the present methods and compositions. In certain embodiments, for example, the promoter is a constitutive promoter, an inducible promoter, or a repressible promoter. Examples of promoters include the CMV promoter.

An endogenous promoter is one that is naturally associated with a gene or sequence. Certain advantages are gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™ (see U.S. Pat. Nos. 4,683,202 and 5,928,906, each incorporated herein by reference).

Naturally, it will be important to employ a promoter and/or enhancer that effectively directs the expression of the DNA segment in the complementing cell. Those of skill in the art of molecular biology generally understand the use of promoters, enhancers, and cell type combinations for protein expression, for example, see Sambrook et al. (2001), incorporated herein by reference.

The particular promoter that is employed to control the expression of the nucleic acid of interest is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used. The use of other viral or mammalian cellular or bacterial phage promoters well-known in the art to achieve expression of polynucleotides is contemplated as well, provided that the levels of expression are sufficient to produce an complementing cell line. Additional examples of promoters/elements that may be employed, in the context of the present invention include the following, which is not intended to be exhaustive of all the possible promoter and enhancer elements, but, merely, to be exemplary thereof.

Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990); Immunoglobulin Light Chain (Queen et al., 1983; Picard et al., 1984); T Cell Receptor (Luria et al., 1987; Winoto et al., 1989; Redondo et al.; 1990); HLA DQ a and/or DQ βSullivan et al., 1987); β Interferon (Goodbourn et al., 1986; Fujita et al., 1987; Goodbourn et al., 1988); Interleukin-2 (Greene et al., 1989); Interleukin-2 Receptor (Greene et al., 1989; Lin et al., 1990); MHC Class II (Koch et al., 1989); MHC Class II HLA-DRa (Sherman et al., 1989); β-Actin (Kawamoto et al., 1988; Ng et al.; 1989); Muscle Creatine Kinase (MCK) (Jaynes et al., 1988; Horlick et al., 1989; Johnson et al., 1989); Prealbumin (Transthyretin) (Costa et al., 1988); Elastase I (Omitz et al., 1987); Metallothionein (MTII) (Karin et al., 1987; Culotta et al., 1989); Collagenase (Pinkert et al., 1987; Angel et al., 1987); Albumin (Pinkert et al., 1987; Tronche et al., 1989, 1990); α-Fetoprotein (Godbout et al., 1988; Campere et al., 1989); t-Globin (Bodine et al., 1987; Perez-Stable et al., 1990); β-Globin (Trudel et al., 1987); c-fos (Cohen et al., 1987); c-HA-ras (Triesman, 1986; Deschamps et al., 1985); Insulin (Edlund et al., 1985); Neural Cell Adhesion Molecule (NCAM) (Hirsh et al., 1990); α1-Antitrypsin (Latimer et al., 1990); H2B (TH2B) Histone (Hwang et al., 1990); Mouse and/or Type I Collagen (Ripe et al., 1989); Glucose-Regulated Proteins (GRP94 and GRP78) (Chang et al., 1989); Rat Growth Hormone (Larsen et al., 1986); Human Serum Amyloid A (SAA) (Edbrooke et al., 1989); Troponin I (TN I) (Yutzey et al., 1989); Platelet-Derived Growth Factor (PDGF) (Pech et al., 1989); Duchenne Muscular Dystrophy (Klamut et al., 1990); SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988); Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell and/or Villarreal, 1988); Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989); Papilloma Virus (Campo et al., 1983; Lusky et al., 1983; Wilkie, 1983; Spalholz et al., 1985; Lusky et al., 1986; Cripe et al., 1987; Gloss et al., 1987; Hirochika et al., 1987; Stephens et al., 1987); Hepatitis B Virus (Bulla et al., 1986; Jameel et al., 1986; Shaul et al., 1987; Spandau et al., 1988; Vannice et al., 1988); Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989); Cytomegalovirus (CMV) (Weber et al., 1984; Boshart et al., 1985; Foecking et al., 1986); Gibbon Ape Leukemia Virus (Holbrook et al., 1987; Quinn et al., 1989).

Enhancers were originally detected as genetic elements that increased transcription from a promoter located at a distant position on the same molecule of DNA. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have very similar modular organization. Additionally, any promoter/enhancer combination (as per the Eukaryotic Promoter Data Base EPDB) could also be used to drive expression of a gene. Further selection of a promoter that is regulated in response to specific physiologic signals can permit inducible expression of a construct. For example, with the polynucleotide under the control of the human PAI-1 promoter, expression is inducible by tumor necrosis factor. Examples of inducible elements, which are regions of a nucleic acid sequence that can be activated in response to a specific stimulus include (Element/Inducer): MT II/Phorbol Ester (TFA) or Heavy metals (Palmiter et al., 1982; Haslinger et al., 1985; Searle et al., 1985; Stuart et al., 1985; Imagawa et al., 1987, Karin et al., 1987; Angel et al., 1987b; McNeall et al., 1989); MMTV (mouse mammary tumor virus)/Glucocorticoids (Huang et al., 1981; Lee et al., 1981; Majors et al., 1983; Chandler et al., 1983; Ponta et al., 1985; Sakai et al., 1988); β-Interferon/poly(rI)x or poly(rc) (Tavernier et al., 1983); Adenovirus 5 E2/E1A (Imperiale et al., 1984); Collagenase/Phorbol Ester (TPA) (Angel et al., 1987a); Stromelysin/Phorbol Ester (TPA) (Angel et al., 1987b); SV40/Phorbol Ester (TPA) (Angel et al., 1987b); Murine MX Gene/Interferon, Newcastle Disease Virus (Hug et al., 1988); GRP78 Gene/A23187 (Resendez et al., 1988); α-2-Macroglobulin/IL-6 (Kunz et al., 1989); Vimentin/Serum (Rittling et al., 1989); MHC Class I Gene H-2κb/Interferon (Blanar et al., 1989); HSP70/E1A, SV40 Large T Antigen (Taylor et al., 1989, 1990a, 1990b); Proliferin/Phorbol Ester-TPA (Mordacq et al., 1989); Tumor Necrosis Factor/PMA (Hensel et al., 1989); and Thyroid Stimulating Hormone α Gene/Thyroid Hormone (Chatterjee et al., 1989).

### II. USE OF E1-COMPLEMENTING CELLS IN PRODUCTION E1-DELETED ADENOVIRUS

The E1-complementing cells of the invention are useful for a variety of purposes. Typically, the cells are used in packaging recombinant virus (i.e., viral particles) from E1-defective vectors and in production of E1-defective adenoviruses in the absence of detectable RCA.

The cells of the invention which express Ad5 E1a and E1b are suitable for use in packaging recombinant virus from E1-defective vectors (e.g., plasmids) containing sequences of Ad5 and Ad2. Further, these cells are anticipated to be useful in producing recombinant virus from other Ad serotypes, which are known to those of skill in the art.

### A. Packaging of E1-Defective Vectors

In certain embodiments, this method of the invention involves packaging of an E1-deleted vector containing a transgene into an E1-deleted adenoviral particle useful for delivery of the transgene to a host cell. In certain aspects, the E1-deleted vector contains all other adenoviral genes necessary to produce and package an infectious adenoviral particle which replicates only in the presence of complementing E1 proteins, e.g., such as are supplied by cell line of the invention. The vector contains defects in the E1a and/or E1b sequences, and most desirably, is deleted of all or most of the sequences encoding these proteins.

At a minimum, the E1-deleted vector to be packaged contains adenoviral 5′ and 3′ cis-elements necessary for replication and packaging, and a transgene. The vector further contains regulatory sequences which permit expression of the encoded transgene product in a host cell, such regulatory sequences are operably linked to the transgene. As used herein, “operably linked” sequences include both expression control sequences that are contiguous with the gene of interest and expression control sequences that act in trans or at a distance to control the gene of interest. Also included in the vector are regulatory sequences operably linked to other gene products carried by the vector.

1. Adenoviral Elements

Typically, the E1-defective vector to be packaged includes adenovirus cis-acting 5′ and 3′ inverted terminal repeat (ITR) sequences of an adenovirus (which function as origins of replication) and the native 5′ packaging/enhancer domain. These are 5′ and 3′ cis-elements used for packaging linear Ad genomes and further contain the enhancer elements for the E1 promoter.

The E1-deleted vector to be packaged into a viral particle may be further engineered so that it expresses the pIX gene product. Most suitably, the pIX gene is intact, containing the native promoter and encoding the full length protein. However, were desired, the native pIX promoter may be substituted by another desired promoter. Alternatively, sequences encoding a functional fragment of pIX may be selected for use in the vector. In yet another alternative, the native sequences encoding pIX or a functional fragment thereof may be modified to enhance expression. For example, the native sequences may be modified, e.g., by site-directed mutagenesis or another suitable technique, to insert preference codons to enhance expression in a selected host cell. Optionally, the pIX may be supplied to the E1-complementing cell line on a separate molecule. This expression of pIX by the vector is contemplated to enhance the pIX expression of complementing cell lines.

An example of a vector containing only the minimal adenoviral sequences is termed the AdΔE1-E4 vector, and lacks all functional adenoviral genes including E1, E2, E3, E4, intermediate gene IXa and late genes L1, L2, L2, L4 and L5) with the exception of intermediate gene IX which is present. In another aspect, a vector can be the Advexin™ vector (Introgen Therapeutic, Inc., Houston, Tex.). In other aspects, the E1-deleted vector contains, in addition to the adenoviral sequences described above, functional adenoviral E2 and E4 regions. In another suitable embodiment, the adenoviral sequences in the E1-deleted vector include the 5′ and 3′ cis-elements, functional E2 and E4 regions, intermediate genes IX and IXa, and late genes L1 through L5. In still further aspects, the E1-deleted vector may be readily engineered by one of skill in the art, taking into consideration sequences required, and is not limited to these examples.

The vector is constructed such that the transgene and/or the sequences encoding pIX are located downstream of the 5′ ITRs and upstream of the 3′ ITRs. The transgene is a nucleic acid sequence, heterologous to the adenovirus sequence, which encodes a polypeptide, protein, or other product, of interest. The transgene is operatively linked to regulatory components in a manner which permits transgene transcription.

2. Transgene

The composition of the transgene sequence will depend upon the use to which the resulting virus will be put. For example, one type of transgene sequence includes a reporter sequence, which upon expression produces a detectable signal. Such reporter sequences include without limitation, DNA sequences encoding β-lactamase, β-galactosidase (LacZ), alkaline phosphatase, thymidine kinase, fluorescent protein (such as green fluorescent protein (GFP)), chloramphenicol acetyltransferase (CAT), luciferase, membrane bound proteins including, for example, CD2, CD4, CD8, the influenza hemagglutinin protein, and others well known in the art to which high affinity antibodies directed thereto exist or can be produced by conventional means, methods of detection are well known, and/or fusion proteins comprising a membrane bound protein appropriately fused to an antigen tag domain from, among others, hemagglutinin or Myc.

In certain aspects the transgene is a non-marker sequence encoding a product which is useful in biology and medicine, such as proteins, peptides, anti-sense nucleic acids (e.g., RNAs), antigens, enzymes, or catalytic RNAs. The transgene may be used to correct or ameliorate gene deficiencies, which may include deficiencies in which normal genes are expressed at less than normal levels or deficiencies in which the functional gene product is not expressed. One desirable type of transgene sequence encodes a therapeutic protein or polypeptide which is expressed in a host cell. The invention further includes using multiple transgenes, e.g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., total of the DNA encoding the subunits and the IRES is less than five kilobases. Other useful gene products include, molecules which induce an immune response, non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a gene. However, the selected transgene may encode any product desirable for delivery to a host or desirable for study. The selection of the transgene sequence is not a limitation of this invention.

In certain aspects, an adenovirus may comprise a transgene encoding a p53, MDA-7, PTEN, FUS-1 or FHIT polypeptide. As used in this application, the term “transgene” refers to a polynucleotide of greater than 10 nucleotides. Therefore, a “transgene encoding a p53, MDA-7, PTEN, FUS1 or FHIT” refers to a DNA segment that encodes p53, MDA-7, PTEN, FUS1, FHIT, or other therapeutic polypeptide or polynucleotide. Similarly, a polynucleotide comprising an isolated or purified transgene, such as a p53, MDA-7, PTEN, FUS1 or FHIT transgene refers to a DNA segment including p53, MDA-7, PTEN, FUS1 or FHIT polypeptide coding sequences and, in certain aspects, regulatory sequences, isolated substantially away from other naturally occurring genes or protein encoding sequences. In this respect, the term “transgene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid encoding a therapeutic polynucleotide, e.g., p53, MDA-7, PTEN, FUS-1 or FHIT may contain a contiguous polynucleotide sequence encoding all or a portion of a therapeutic polypeptide, e.g., p53, MDA-7, PTEN, FUS-1 or FHIT, of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, or more nucleotides or base pairs.

a. Therapeutic Polynucleotides

A therapeutic polynucleotide of the invention typically falls into one of three categories; pro-drug converting enzyme for suicide gene therapy, cytokine gene to augment anti-tumor immune responses, or pro-apoptotic protein or polynucleotide. Although, other therapeutic polynucleotides such as anti-sense RNA, miRNA, and siRNA are also contemplated, particularly when the down regulation of a growth promoting gene is desired.

b. Inhibitors of Cellular Proliferation

Tumor suppressors function to inhibit excessive cellular proliferation. The inactivation of these genes destroys their inhibitory activity, resulting in unregulated proliferation. Non-limiting examples of transgenes that may be employed according to the present invention include Rb, p16, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC (mutated in colorectal cancer).

c. Regulators of Programmed Cell Death

Apoptosis, or programmed cell death, is an essential process for normal embryonic development, maintaining homeostasis in adult tissues, and suppressing carcinogenesis (Kerr et al., 1972). Non-limiting examples of these proteins that may be employed according to the present invention include different family members having similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

d. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. However, oncogenes rarely arise from genes encoding growth factors. In one embodiment of the present invention, it is contemplated that anti-sense mRNA or siRNA directed to a particular inducer of cellular proliferation may be used to prevent expression of the inducer of cellular proliferation. Targets or transgene can include the proteins FMS, ErbA, ErbB, Src, Abl, and/or Ras.

e. Antigens and Vaccines

The present invention can also be used with vectors, compositions and methods useful for vaccination. The antigen can be presented in the adenovirus capsid, alternatively, the antigen can be expressed from a heterologous nucleic acid introduced into a recombinant adenovirus genome. Any immunogen of interest can be provided by the adenovirus vector.

The Adenoviral vaccine of the invention includes at least one, two, three or more nucleotide coding regions, each coding region encoding an immunogenic polypeptide component. The coding regions may be in the same or different adenoviral vector, each of which may be a RD or RC Ad. When it contains two or more nucleotide coding regions, the polynucleotide vaccine is referred to herein as a “multicomponent” polynucleotide vaccine.

In addition, the vector construct can contain nucleotide sequences encoding cytokines, such as granulocyte macrophage colony stimulating factor (GM-CSF), interleukin-12 (IL-12) and co-stimulatory molecules such B7-1, B7-2, CD40. The cytokines can be used in various combinations to fine-tune the response of the subject's immune system, including both antibody and cytotoxic T lymphocyte responses, to bring out the specific level of response needed to control or eliminate the infection or disease state.

Diseases against which a subject may be immunized include viral diseases, allergic manifestations, diseases caused by bacterial or other pathogens, such as parasitic organisms, AIDS, autoimmune diseases such as Systemic Lupus Erythematosus, Alzheimer's disease and cancers. Suitable antigens comprise bacterial, viral, fungal and protozoan antigens derived from pathogenic organisms, as well as allergens, and antigens derived from tumors and self-antigens. Typically, the antigen will be a protein, polypeptide or peptide antigen.

Specific examples of antigens useful in the present invention include a wide variety of proteins from the herpesvirus family, including proteins derived from herpes simplex virus (HSV) types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH; and antigens derived from other human herpesviruses such as HHV6 and HHV7. (See, e.g. Chee et al., Cytomegaloviruses (McDougall, 1990) for a review of the protein coding content of cytomegalovirus; McGeoch et al. (1988), for a discussion of the various HSV-1 encoded proteins; U.S. Pat. No. 5,171,568 for a discussion of HSV-1 and HSV-2 gB and gD proteins and the genes encoding therefor; Baer et al. (1984), for the identification of protein coding sequences in an EBV genome; and Davison and Scott (1986), for a review of VZV.)

Antigens derived from other viruses will also find use in the inventive methods, such as without limitation, proteins from members of the families Picornaviridae (e.g., polioviruses, etc.); Caliciviridae; Togaviridae (e.g., rubella virus, dengue virus, etc.); Flaviviridae; Coronaviridae; Reoviridae; Birnaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae (e.g., HTLV-I; HTLV-II; HIV-1 (also known as HTLV-III, LAV, ARV, hTLR, etc.)), including but not limited to antigens from the isolates HIV (III)b′ HIV (SF2), HIV (LAV), HIV (LAI), HIV (MN)); HIV-1 (CM235), HIV-1 (US4); HIV-2; simian immunodeficiency virus (SIV) among others. See, e.g. Joklik, 1988); Fields and Knipe (1991), for a description of these and other viruses.

Additionally, the envelope glycoproteins HA and NA of influenza A are of particular interest for generating an immune response. Numerous HA subtypes of influenza A have been identified (Kawaoka et al., 1990; Webster et al., 1983. Thus, proteins derived from any of these isolates can also be used in the techniques described herein.

The compositions and methods described herein will also find use with numerous bacterial antigens, such as those derived from organisms that cause diphtheria, cholera, tuberculosis, tetanus, pertussis, meningitis, and other pathogenic states, including, without limitation, Bordetella pertussis, Neisseria meningitides (A, B, C, Y), Hemophilus influenza type B (HIB), and Helicobacter pylori. Examples of parasitic antigens include those derived from organisms causing malaria and Lyme disease.

3. Regulatory Sequences

In addition to the major elements identified above for the vector, (e.g., the adenovirus sequences and the transgene), the vector also includes conventional control elements necessary to drive expression of the adenoviral genes and/or transgene in a host cell. Thus the vector contains a selected promoter which is linked to the genes or transgene and located between the viral sequences of the vector to be packages or integrated into the complementing cell. Suitable promoters may be readily selected from among constitutive and inducible promoters. Selection of these and other common vector elements are conventional and many such sequences are available [see, e.g., Sambrook et al, and references cited therein].

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [e.g., Boshart et al., Cell, 41:521-530 (1985)], the SV40 promoter, the dihydrofolate reductase promoter, the β-actin promoter, the phosphoglycerol kinase (PGK) promoter, and the EF1 promoter.

Inducible promoters are regulated by exogenously supplied compounds, including, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al., Proc. Natl. Acad. Sci. USA, 93:3346-3351 (1996)], the tetracycline-repressible system [Gossen et al., Proc. Natl. Acad. Sci. USA, 89:5547-5551 (1992)], the tetracycline-inducible system [Gossen et al., Science, 268:1766-1769 (1995), see also Harvey et al., Curr. Opin. Chem. Biol., 2:512-518 (1998)], the RU486-inducible system [Wang et al., Nat. Biotech., 15:239-243 (1997) and Wang et al., Gene Ther., 4:432-441 (1997)] and the rapamycin-inducible system [Magari et al., J. Clin. Invest., 100:2865-2872 (1997)].

4. Other Vector Elements

The vector carrying the Ad ITRs flanking the transgene and regulatory sequences (e.g., promoters, polyA sequences, etc.) may be in any form which transfers these components to the host cell. Preferably, the vector is in the form of a plasmid. Typically, to avoid homologous recombination, the plasmid does not contain any adenovirus sequences in the E1 region or the region 5′ to the E1 region. It may contain non-viral sequences, such as those encoding certain selectable reporters or marker genes, e.g., sequences encoding hygromycin or purimycin, among others. Other components of the plasmid may include an origin of replication and an amplicon, such as the amplicon system, employing the Epstein Barr virus nuclear antigen, for example, the vector components in pCEP4 (Invitrogen). See, also, J. Horvath et al., Virology, 184:141-148 (1991). This amplicon system or similar amplicon components permit high copy episomal replication in the cells.

Other heterologous nucleic acid sequences optionally present in this vector include sequences providing signals required for efficient polyadenylation of the RNA transcript, and introns with functional splice donor and acceptor sites. A common poly-A sequence which is employed in the vectors is that derived from the papovavirus SV-40. The poly-A sequence generally is inserted following the transgene sequences and before the 3′ AAV ITR sequence. A vector useful in the present invention may also contain an intron, desirably located between the promoter/enhancer sequence and the transgene. One possible intron sequence is also derived from SV-40, and is referred to as the SV-40 T intron sequence. Selection of these and other common vector elements are conventional and many such sequences are available (see, e.g., Sambrook et al., and references cited therein).

5. Co-Transfection of Adenoviral Sequences

Typically, the E1-deleted vector to package in the complementing cells of the invention contain all functional adenoviral sequences required for packaging and replication in the presence of the E1-complementing cell line of the invention. In addition to the E1a and E1b functions supplied by the trans-complementing cell line, functional adenoviral E2a and E4 ORF 6 region are required. However, where the required functions are lacking from the E1-deleted vector (i.e., the E1-deleted vector further contains functional deletions in E2a and/or E4 ORF6), these functions may be supplied by other sources or the complementing cell lines. In one embodiment, these functions may be supplied by co-transfection of the E1-complementing cell line with one or more nucleic acid molecules capable of directing expression of the required adenoviral function. Alternatively, a modified cell line(s) of the invention that has been transformed to supply the required adenoviral functions may be utilized.

For example, a vector deleted of E1 and having a defective E2 region may be complemented in cells of the invention by transfecting the cells with a nucleic acid molecule (e.g., a plasmid or expression cassette) expressing required E2 functions (e.g., E2a). As another example, a vector lacking E1 through E4 functions may be complemented in cells by transfecting the cells with a nucleic acid molecule expressing functional E2, E3 and E4 (e.g., E4 ORF6). Where a nucleic acid molecule is co-transfected into the cells of the invention, such a nucleic acid molecule typically contains no adenoviral E1 sequences; nor does it contain any sequences 5′ to the E1 region. Construction of these nucleic acid molecules is within the skill of those in the art.

A selected recombinant vector, as described above, is introduced into E1-complementing cells from a cell line of the invention using conventional techniques, such as the transfection techniques known in the art [see, K. Kozarsky et al., Som. Cell and Molec. Genet., 19(5):449-458 (1993)]. Thereafter, recombinant E1-deleted adenoviruses are isolated and purified following transfection. Purification methods are well known to those of skill in the art and may be readily selected. For example, the viruses may be subjected to plaque purification and the lysates subjected to cesium chloride centrifugation to obtain purified virus.

### III. AMPLIFICATION OF E1-DELETED ADENOVIRUSES

The E1-trans-complementing cell line of the invention (or a derivative thereof) may be used to amplify an E1-defective adenovirus. Suitably, the E1-defective adenovirus will have been isolated and purified from cellular debris and other viral materials prior to use. Suitable purification methods, e.g., plaque purification, chromatographic purifications etc., are well known to those of skill in the art.

Typically, a culture, which includes a cell suspension, from an E1-trans-complementing cell line of the invention is infected with the E1-defective adenovirus using conventional methods. A suitable multiplicity of infection (MOI) may be readily selected. However, an MOI in the range of about 0.1 to about 100, about 0.5 to about 20, and/or about 1 to about 5, can be used. The cells are then cultured under conditions that permits cell growth and replication of the E1-defective adenovirus in the presence of the E1 expressed by the cell line of the invention. The viruses can be subjected to continuous passages for up to 5, 10, or 20 passages or more including all values and ranges there between. Where desired the viruses may be subjected to fewer or more passages.

In certain embodiments, the cells are subjected to two to three rounds of freeze-thawing, the resulting lysate is then subjected to centrifugation for collection, and the supernatant is collected. Conventional purification techniques such as chloride gradient centrifugation or column chromatography are used to concentrate the recombinant E1-defective adenovirus (rAdΔE1) from the cellular proteins in the lysate. The compositions and methods of the invention can avoid or reduce the problems of contaminating RCA associated with conventional production techniques.

### A. E1-Deleted Ad Produced by Method of Invention

The E1-deleted adenoviruses produced according to the present invention are suitable for a variety of uses and are particularly suitable for in vivo use, as the present invention enables these adenoviruses to be produced in serum-free media, and in the absence of detectable RCA. Thus, the E1-deleted adenoviruses produced according to the invention are substantially free of contamination with RCA.

In certain aspects, E1-deleted viruses have been deemed suitable for applications in which transient transgene expression is therapeutic (e.g., p53 gene transfer in cancer). The E1-deleted adenoviruses are not limited to use where transient transgene expression is desired. The E1-deleted adenoviruses are useful for a variety of situations in which delivery and expression of a selected transgene is desired.

Suitable doses of E1-deleted adenoviruses may be readily determined by one of skill in the art, depending upon the condition being treated, the health, age and weight of the veterinary or human patient, and other related factors. However, generally, a suitable dose may be in the range of about 10¹⁰ to 10¹⁸ or about 10¹² to 10¹⁶ viral particles per dose, for an adult human having weight of about 80 kg. This dose may be suspended in about 0.01 mL to about 1 mL of a physiologically compatible carrier and delivered by any suitable means. The dose may be repeated, as needed or desired, daily, weekly, monthly, or at other selected intervals.

Embodiments of the invention include an expression cassette construct and a cell line harboring such expression cassette for the production of commercial-grade adenoviral products. The related biological manufacturing systems can be integrated with product safety attributes and commercial scale production. The compositions of the invention will typically include cell lines to generate a safe and economical adenovirus-based product and include adenoviral compositions that are (1) traceable, (2) free of animal-derived components, (3) scalable, and/or (4) free of replication-competent adenovirus (RCA). Cell lines of the invention may be use in producing a variety of commercial biologics including vaccines, therapeutic proteins, and antibodies.

The use of protein IX in both the expression cassette and the adenoviral vector will allow for improved virus yield and stability over existing cell lines. However, the lack of homology between the expression cassette and the adenoviral vector DNA will eliminate the possibility of a recombination to generate a replication competent adenovirus.

### IV. EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Production of Complementing Cell Lines

Selection and isolation of cell lines. An initial screen of the potential cells or cell lines is performed. Cells that meet the criteria for both growth and usefulness for adenovirus production are isolated and chosen to move forward to the next segment of the activities.

Expression cassette construction and transfection. An E1 gene expression cassette that does not have sequence homology with available E1 deleted adenovirus vectors is constructed. The E1 expression cassette is introduced into a subset of the evaluated lines.

Evaluation of cell lines. Cells isolated and subsequently transfected with the expression cassette described above are evaluated for their ability to amplify adenovirus. The E1 transfected cells will be evaluated for E1 protein expression and stability. A small number of stable cell lines are chosen to move forward to the next segment of the activities.

Optimization of selected cell lines. A series of ranging studies (temperature, multiplicity of infection, etc.) are performed on the cell lines proceeding through the studies in order to further evaluate the potential of each of the potential lines for RCA-free production of adenovirus vector. A final cell line is selected and banked for further characterization study.

Characterization and Comparability of Chosen Cell Line. After choosing of a cell line, a full characterization study is performed to ascertain the properties of the line, and to compare the adenovirus output relative to the lines in current use (such as HeLa and 293 cells).

Adherent and Suspension Cell Processes. Modifying adenovirus production process to remove all animal-sourced components is one goal of the invention. Typically, clinical products have relied upon one or more animal-sourced components within the process, as adherent cell lines have required the use of both fetal calf serum and porcine-derived trypsin. Manufacturing processes can be developed that eliminate all animal-sourced components by the development of a process based upon the use of suspension cell culture for the amplification of adenovirus. One such process uses the Wave Bioreactor systems and HEK 293 cell line that has been adapted to suspension culture in a protein-free medium (referred to as the “suspension process”). In concert with the change to the cell culture method, additional changes were made to streamline the manufacturing process and to improve the production method for the final drug product. These changes are described in the following sections.

### Example 2

### Manufacturing Process

Cells and Master Cell Bank. Initially, adenovirus production utilized HEK 293 cells manufactured in a CellCube manufacturing process. The Cell Banks received extensive characterization as part of their release testing in accordance with the existing FDA guidance documents and ICH guidelines. The suspension production process utilizes a modified lineage of HEK 293 cells adapted to a protein-free medium. The cell line was obtained from Invitrogen and in combination with Invitrogen's protein-free medium, CD293, gave excellent results both in terms of supporting cell growth and amplification in several adenoviral products.

Suspension HEK 293 Lineage. The immediate source of the cells used to establish the suspension-cell master cell bank (MCB) was Invitrogen catalog number 11631-017 293-F. These cells are descended from the parental 293 cell line established by Dr. Frank Graham of McMaster Carr University of Toronto, Canada.

Master Cell Bank Testing. The new MCB adds specific tests to increase the assurance of freedom from adventitious agents and to better characterize the bank viability, growth, and viral amplification characteristics. Tests include: Bovine, Porcine and Equine viruses; Hepatitis A; HHV 7 and 8; Polyoma virus BKV and JCV; cell growth characterization and viral acceptability. Specifications for the original panel of tests remain the same. A comparison of the testing performed can found in the following table:

| | Adherent | Suspension | |
|---|---|---|---|
| Test Name | MCB | MCB | Change |
| Viability (Trypan Blue | Yes | Yes | — |
| Exclusion) | | | |
| Sterility | Yes | Yes | — |
| Mycoplasma (PTC) | Yes | Yes | Added EP component |
| Bacteriostasis/Fungistasis | Yes | Yes | — |
| Product Enhanced | Yes | Yes | — |
| Reverse Transcriptase | | | |
| (PERT) Assay | | | |
| In vitro Assay for | Yes | Yes | — |
| Adventitious Viruses | | | |
| In vivo Assay for | Yes | Yes | — |
| Inapparent Viruses | | | |
| Transmission Electron | Yes | Yes | — |
| Microscopy (TEM) | | | |
| Karyology and | Yes | Yes | — |
| Isoenzyme Analysis | | | |
| B19 | Yes | Yes | — |
| AAV | Yes | Yes | — |
| EBV | Yes | Yes | — |
| CMV | Yes | Yes | — |
| HBV | Yes | Yes | — |
| HCV | Yes | Yes | — |
| HIV1 and 2 | Yes | Yes | — |
| HTLV1 and 2 | Yes | Yes | — |
| HHV6 | Yes | Yes | — |
| In vitro Assay for the | No | Yes | New for Suspension MCB |
| Presence of Bovine | | | |
| Viruses | | | |
| In vitro Assay for the | No | Yes | New for Suspension MCB |
| Presence of Porcine | | | |
| Viruses | | | |
| In vitro Assay for the | No | Yes | New for Suspension MCB |
| Presence of Equine | | | |
| Viruses | | | |
| HHV7 | No | Yes | New for Suspension MCB |
| HHV8 | No | Yes | New for Suspension MCB |
| HAV | No | Yes | New for Suspension MCB |
| Polyoma Virus BKV and | No | Yes | New for Suspension MCB |
| JCV | | | |
| Cell Growth | No | Yes | New for Suspension MCB |
| Characterization | | | |
| Viral Acceptability | No | Yes | New for Suspension MCB |

Serum and Protein Free Media. The media used for the adherent process was Dulbecco's Modified Eagle Medium (DMEM) containing 10% fetal calf serum. The adherent nature of the cell culture process also required the use of porcine derived trypsin for cell detachment purposes. The suspension culture process removes both the media containing fetal calf serum and the porcine-derived trypsin. A customized version of Invitrogen's CD293 media is used throughout the cell and viral culture process.

Wave Bioreactor. The expansion process for adherent production process lots utilized T-150 flasks, Cell Factory 10 Units, and the CellCube 4×100 system. These culture vessels for the adherent-cell based process are replaced by shaker flasks of increasing sizes to provide an inoculum for a WAVE20 Bioreactor. The 10 L cell culture in the WAVE20 is perfused with CD293 media to increase biomass to a point that permits the inoculation of a WAVE200 bioreactor. The cells are grown for a further period in the WAVE200 (containing 100 L of CD293 medium) and the culture is then infected with the Adenoviral construct.

The WAVE bioreactor system uses disposable plastic bags (Cellbags) as the cell culture vessel with temperature control provided by a heating pad upon which the Cellbag rests. Gas transfer is mediated through the rocking motion of the heating pad and the passage of gasses above the surface of the medium. The bioreactor system provides a homogeneous environment that can be monitored for pH, temperature and dissolved oxygen. The system provides a more controlled environment than the intrinsically heterogeneous environments produced by adherent cell culture systems. The old process allowed cells to autolyse to release viral particles. An improvement applied to the current process, detergent lysis, has been adopted for current product production which reduces process times by approximately three days.

Current Rationale for RCA Specification. A RCA specification of ≧4 RCA in 3×10¹⁰ viral particles (vp) has been proposed adenovirus products. The level of RCA is intrinsic to the adenoviral construct and the 293 cell line due to the amount of homology between flanking regions of the adenoviral construct and the transcomplementing E1 sequence of the 293 cell line. The construction of follow up adenoviral products at have been performed using the same method as that utilized for Advexin (Introgen's adenoviral p53 product). Similar levels of RCA are to be expected for these products. Based upon the extensive safety data accumulated from administration of Advexin to over 500 patients, FDA acceptance has been received for the RCA specification in follow up products.

The actual level of RCA in the Advexin product is estimated as 1.3 RCA per 3×10¹⁰ vp, based on the assay results from approximately 30 batches and a poisson distribution. The value of ≦4 allows the assay method to be interpreted correctly, as a result of 3 or 4 is possible based on the poisson distribution even if the amount of RCA in a given batch is actually only 1 or 2. This is a result of the current assay design which tests the product at near the limit of quantitation of its method. Therefore, the proposed specification of ≦4 RCA in 3×10¹⁰ vp is seen as acceptably rigid based on both the intrinsic content of RCA within the product and the assay method usage at near the limit of quantitation.

### Example 3

### Research Design and Methods

A suspension process allows for the elimination of animal-derived components, principally in the media sera, and for greater scalability. While the process change has yielded a safer and more robust production process, attributes such as traceability and RCA remain uncertain.

Selection and isolation of cell lines. One aspect of the invention involves the selection and isolation of primary cells from human tonsil and/or umbilical cord. The tonsil and umbilical cord cells are selected due to their high growth rate and utility in cell culture and susceptibility to adenovirus infection. Viable cell lines for testing and comparison to HEK 293 and HeLa suspension cell lines are contemplated.

Primary cell line selection. Tonsil and umbilical cord primary cells may be procured through a hospital affiliate. All primary cells are collected upon consent from young, healthy volunteers with no significant family history of cancer or heart disease.

Isolation of primary cell lines. Cells are isolated from the collected tissue samples after enzymatic treatment. Cells are cultured and selected for population with the epithelial morphology in an appropriate media to remove contaminating fibroblast cells. The selected primary cells are further expanded for frozen storage. All raw materials used are typically traceable. At least 3 cell lines each for tonsil umbilical groups are isolated. The selected cell lines are chosen based on growth rate and robustness.

Expression cassette construction and transfection. Construction of an expression cassette and subsequent transfection of cell lines include expression cassettes that reduce or eliminate RCA production and/or include expression regions for production of protein IX. It is contemplated that protein IX encoding sequence are can be expressed both by the cell line and packaged vector to improved virus yield and stability.

| | |
|---|---|
| Cell Line | Vector |

| | Canji | Crucell | Canji | Crucell |
|---|---|---|---|---|
| Prom | − | − | + | + |
| E1a | + | + | − | − |
| E1b | + | + | − | − |
| E2 | − | − | + | + |
| E3 | − | − | + | + |
| pIX | + | − | − | + |

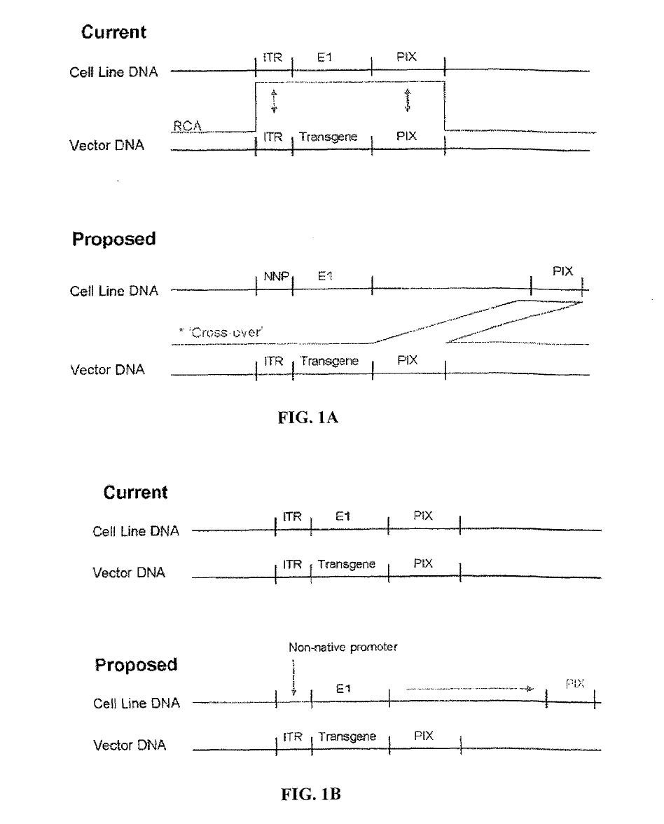

Expression cassette design. Based upon the criteria to design a cell line an expression cassette is designed that produces an adenovirus that is traceable, free of animal-derived components, scalable, and/or free of RCA. Protein IX sequence is one component that can be used to increase stability of the adenovirus product. However, homology between the cell line and vector DNA at the promoter and protein IX gene has been instrumental in the production of RCA. The elimination of RCA is one safety concern with so one or more of the following strategies may be used to reduce or eliminate RCA: (1) replacement of the Inverted Terminal Repeat (ITR) in the cell line with a non-native promoter, (2) locate the protein IX sequence in position that hinders recombination in the cell line avoiding alignment of sequences by inserting a non-coding stuffer sequence between the E1 and protein IX sequence, and/or (3) engineer the sequence of protein IX in the cell line to yield a functional but different protein IX gene, thus reducing homology at the nucleic acid sequence level. As shown in FIG. 1, cross-over events happen do not produce E1 recombination over the transgene and therefore no RCA. Further, the sequence changes in protein IX should further reduce, if not entirely eliminate, any presence of cross-over or RCA.

Expression cassette transfection. The expression cassette is cloned into a high yield plasmid backbone with an appropriate selection marker for amplification and purification. The purified plasmid is used to transfect primary cell lines as well as HeLa suspension cell lines to generate E1 complementing cell lines. Transfection is performed using any one of the standard transfection methods such as the liposome based transfection methods. After transfection, cells are selected for the presence of the expression cassette using the selection marker.

In certain aspects, 3 tonsil and 3 umbilical primary cell lines as well as 1 cancer cell lines (HeLa) transfected with expression cassette are isolated. These cell lines are evaluated and characterized as described below.

Evaluation of cell lines. Evaluation of transfected cell lines for the production of adenoviral based products includes evaluation of (A) Viral productivity, (B) Stability, (C) Lack of recombination events in amplification, (D) Availability of commercial media and host-cell protein assays useful for cell line, and/or (E) Suspension adaptability. The cell lines are screened based on viral productivity and other factors listed above. Typically, the primary cell lines are targeted for further development, the cancerous cell lines are developed because of their current utility and the improvements made in the cell line construction.

Optimization of selected cell lines. Once cell lines are selected based on general criteria for adenoviral production, adaptability of the cell line to manufacturing processes is assessed. Typically a pre-established series of optimization studies are followed that allow the evaluation of the cell lines' ability to be produced at commercial scale quantities. Cell banks and materials for the manufacture of material under GMP are also produced. A series of optimization studies are conducted to refine the conditions for the selected cell line for production of an adenoviral product. In shaker cultures, the following variables are studied to further define the expected manufacturing process: (a) Multiplicity of Infection (MOI), (b) Cell Density at time of infection, and (c) Infection temperature. The optimization studies are followed by a Wave 20 run utilizing the conditions determined from those studies. Non-GMP Wave 20 scale run are used in the Process Development setting. Cells are cultured and infected with the amplified plaque purified adenoviral construct.

Typically, a Research Cell Bank (RCB) is established during the expansion of the cells for the Wave 20 run. This RCB is intended for use in further manufacturing activities as the cell material used to establish a Master Cell Bank (MCB) during the first GMP production run.

Characterization and Comparability of Cell Lines. Furthermore, the characterization and comparability studies of the selected cell line(s) are performed. Characterization studies allow the assessment of the integrity and sterility of the resultant bank for future use. This analysis is typically needed not only for GMP production but also to ensure the capabilities of the cell line for sterile propagation. Comparability studies assess the physical and chemical functionality of the cell line in comparison to 293 cells. In all, these studies are designed to confirm the creation of a safe and robust cell line for adenovirus manufacturing.

Assays are performed to characterize the Research Cell Bank and the Research Virus Bank that are established. Non-validated development assays are typically used for this activity. Research Cell Bank Characterization includes characterization of one or more of (a) Sterility (USP/EP), (b) Bacteriostasis/Fungistasis Qualification for Sterility Testing, (c) Mycoplasma (PCR), (d) In Vitro Adventitious Virus Testing, and/or (e) Western Blot (using E1 antibody).

Comparability studies are performed to assess the similarity of materials made using newly developed cells versus 293 cells. In addition to the characterization performed above, the following analysis can also be performed: (a) Electron Microscopy, (b) Analytical Ultracentrifugation, (c) Potency Assay, and/or (d) Reverse phase HPLC.

Methods for producing and using a novel cell line for the production of adenoviral-based and other biological medicines is described.

### REFERENCES

The following references, to the extent that they provide exemplary procedural or other details supplementary to those set forth herein, are specifically incorporated herein by reference.
Gao et al., Hum Gene Ther. 2000 Jan. 1; 11(1):213-9.
Graham et al., J. gen. Virol. 1977; 36: 59-72.
Robert et al., Gene Ther. 2001; November; 8(22):1713-20.
Murakami et al., Hum Gene Ther. 2002 May 20; 13(8):909-20.
Fallaux et al., Hum Gene Ther. 1998 Sep. 1; 9(13):1909-17.
Hehir et al., J Virol. 1996 December; 70(12):8459-67.

1. An isolated polynucleotide comprising:
(i) a first DNA segment encoding an adenoviral E1 protein;
(ii) a second DNA segment encoding an adenoviral protein IX; and
(iii) a heterologous DNA spacer positioned between the first and second DNA segment.

2. The polynucleotide of claim 1, wherein the DNA spacer is at least 2 kilobases.

3. The polynucleotide of claim 1, wherein the DNA spacer is at least 5 kilobases.

4. The polynucleotide of claim 1, wherein the DNA spacer is at least 10 kilobases.

5. The polynucleotide of claim 1, wherein the protein IX nucleotide sequence is engineered to reduce crossover rate.

6. The polynucleotide of claim 1, wherein the first DNA segment is operatively coupled to a heterologous promoter.

7. An adenoviral E1 complementing cell line comprising an expression cassette comprising
(i) a first DNA segment encoding an adenoviral E1 protein;
(ii) A second DNA segment encoding an adenoviral protein IX; and
(iii) a heterologous DNA spacer positioned between the first and second DNA segment.

8. The cell line of claim 7, wherein the expression cassette is integrated in the cellular genome.

9. The cell line of claim 7, wherein the first DNA segment encoding the adenoviral E1 protein is operatively coupled to a heterologous promoter.

10. The cell line of claim 7, wherein the second DNA segment encoding the adenoviral protein IX is operatively coupled to a second heterologous promoter.

11. The cell line of claim 10, wherein the second DNA segment is modified to reduced recombination with an adenoviral vector.

12. (canceled)

13. The cell of claim 1, further comprising a recombinant E1 deficient adenoviral vector.

14. A system for propagation of recombinant adenoviral vector comprising:
(a) a culture vessel comprising culture media;
(b) a recombinant E1 deficient adenoviral vector; and
(c) cells from the complementing cell line of claim 7

15. A method for producing an E1 deficient recombinant adenoviral vector comprising:
(a) providing a complementing cell comprising:
(i) a first DNA segment encoding an adenoviral E1 protein;
(ii) a second DNA segment encoding an adenoviral protein IX; and
(iii) a heterologous DNA spacer positioned between the first and second DNA segment; and
(b) introducing an E1 deficient adenoviral nucleic acid into the complementing cell;
(c) culturing the complementing cell; and
(d) harvesting recombinant adenovirus produced from or by the complementing cell.
